(19) United States
(12) Patent Application Publication
(10) Pub. No.: US 2022/0031380 A1
(43) Pub. Date: Feb. 3, 2022

(54) PLUNGING TIP FOR BIPOLAR PENCIL

(71) Applicant: Covidien LP, Mansfield, MA (US)

(72) Inventors: Thomas A. Zammataro, North Haven, CT (US); Brian J. Creston, Madison, CT (US); Jacob C. Baril, Norwalk, CT (US)

(21) Appl. No.: 16/941,006

(22) Filed: Jul. 28, 2020

(51) Int. Cl. A61B 18/14

(52) U.S. Cl. CPC A61B 18/1402; A61B 2018/126

(57) ABSTRACT

An electrode assembly for an electrosurgical instrument includes a housing having an active electrical connector and a return electrical connector configured to operably engage a distal end of an electrosurgical instrument shaft. The housing encapsulates a pair of elongated ground plates and a pair of insulative tubes configured to house first and second ends of a wire-like active electrode. The wire-like active electrode operably couples at the first end to the active electrical connector. The elongated ground plates each include a tip at a distal end thereof configured to mutually support a donut-style insulator configured to support the wire-like active electrode therearound. A tensioning mechanism is configured to operably engage the second end of the wire-like active electrode. The tensioning mechanism cooperates with a tensioning tool to tension the wire-like active electrode about the donut-like insulator during assembly.

### BACKGROUND

### Technical Field

The present disclosure relates generally to electrosurgical instruments and, more particularly, to an electrosurgical bipolar pencil having an end effector assembly with a donut-style plunging tip.

### Background of Related Art

Electrosurgical instruments have become widely used by surgeons in recent years. Accordingly, a need has developed for equipment and instruments which are easy to handle, are reliable and are safe in an operating environment. By and large, most electrosurgical instruments are hand-held instruments, e.g., an electrosurgical pencil, which transfer radio-frequency (RF) electrical or electrosurgical energy to a tissue site. The electrosurgical energy is returned to the electrosurgical source via a return electrode pad positioned under a patient (i.e., a monopolar system configuration) or a smaller return electrode positionable in bodily contact with or immediately adjacent to the surgical site (i.e., a bipolar system configuration). The waveforms produced by the RF source yield a predetermined electrosurgical effect known generally as electrosurgical coagulation, electrosurgical sealing, electrosurgical cutting, and/or electrosurgical fulguration or, in some instances, an electrosurgical blend thereof.

In particular, electrosurgical fulguration includes the application of an electric spark to biological tissue, for example, human flesh or the tissue of internal organs, without significant cutting. The spark is produced by bursts of radio-frequency electrical or electrosurgical energy generated from an appropriate electrosurgical generator. Coagulation is defined as a process of desiccating tissue wherein the tissue cells are ruptured and dehydrated/dried. Electrosurgical cutting/dissecting, on the other hand, includes applying an electrical spark to tissue in order to produce a cutting, dissecting and/or dividing effect. Blending includes the function of cutting/dissecting combined with the production of a hemostasis effect. Meanwhile, sealing/hemostasis is defined as the process of liquefying the collagen in the tissue so that it forms into a fused mass.

As used herein the term “electrosurgical pencil” is intended to include instruments that have a handpiece which is attached to an active electrode and that is used to cauterize, coagulate and/or cut tissue. Typically, the electrosurgical pencil may be operated by a handswitch or a foot switch.

As mentioned above, the handpiece of the electrosurgical pencil is connected to a suitable electrosurgical energy source (e.g., generator) that produces the radio-frequency electrical energy necessary for the operation of the electrosurgical pencil. In general, when an operation is performed on a patient with an electrosurgical pencil in a monopolar mode, electrical energy from the electrosurgical generator is conducted through the active electrode to the tissue at the site of the operation and then through the patient to a return electrode. The return electrode is typically placed at a convenient place on the patient's body and is attached to the generator by a conductive material. Typically, the surgeon activates the controls on the electrosurgical pencil to select the modes/waveforms to achieve a desired surgical effect. Typically, the “modes” relate to the various electrical waveforms, e.g., a cutting waveform has a tendency to cut tissue, a coagulating wave form has a tendency to coagulate tissue, and a blend wave form tends to be somewhere between a cut and coagulate wave from. The power or energy parameters are typically controlled from outside the sterile field which requires an intermediary like a circulating nurse to make such adjustment.

When an operation is performed on a patient with an electrosurgical pencil in a bipolar mode, the electrode face includes at least one pair of bipolar electrodes and electrical energy from the electrosurgical generator is conducted through tissue between the pair of bipolar electrodes.

A typical electrosurgical generator has numerous controls for selecting an electrosurgical output. For example, the surgeon can select various surgical “modes” to treat tissue: cut, blend (blend levels 1-3), low cut, desiccate, fulgurate, spray, etc. The surgeon also has the option of selecting a range of power settings typically ranging from 1-300 W. As can be appreciated, this gives the surgeon a great deal of variety when treating tissue. Surgeons typically follow preset control parameters and stay within known modes and power settings and electrosurgical pencils include simple and ergonomically friendly controls that are easily selected to regulate the various modes and power settings

Electrosurgical instruments are typically configured such that power output can be adjusted without the surgeon having to turn his or her vision away from the operating site and toward the electrosurgical generator.

### SUMMARY

As used herein, the term “distal” refers to the portion that is described which is further from a user, while the term “proximal” refers to the portion that is being described which is closer to a user. The terms “substantially” and “approximately,” as utilized herein, account for industry-accepted material, manufacturing, measurement, use, and/or environmental tolerances. Further, any or all of the aspects and features described herein, to the extent consistent, may be used in conjunction with any or all of the other aspects and features described herein.

Provided in accordance with aspects of the present disclosure is an electrode assembly for an electrosurgical instrument including a housing having an active electrical connector and a return electrical connector configured to operably engage a distal end of an electrosurgical instrument shaft. The housing encapsulates a pair of elongated ground plates and a pair of insulative tubes configured to house first and second ends of a wire-like active electrode. The wire-like active electrode operably couples at the first end to the active electrical connector. The elongated ground plates each include a tip at a distal end thereof configured to mutually support a donut-style insulator configured to support the wire-like active electrode therearound. A tensioning mechanism is configured to operably engage the second end of the wire-like active electrode. The tensioning mechanism cooperates with a tensioning tool to tension the wire-like active electrode about the donut-like insulator during assembly.

In aspects according to the present disclosure, the tensioning mechanism includes a terminal and an anchoring screw, the anchoring screw configured to secure the terminal against the wire-like active electrode once proper tension has been achieved by rotating the tensioning tool during assembly. In other aspects according to the present disclosure, each tip of each ground plate includes an inwardly extending shaft, the inwardly extending shafts of each tip disposed in registration with one another and configured to mutually support the donut-style insulator.

In aspects according to the present disclosure, the pair of ground plates are substantially triangular to expose a portion of the wire-like active electrode for tissue treatment. In other aspects according to the present disclosure, a first insulative tube extends along an entire length of one side of the pair of ground plates to limit exposure of the wire-like active electrode.

In aspects according to the present disclosure, the pair of ground plates are substantially triangular including an elongated side and a hypotenuse and wherein a first insulative tube extends along an entire length of the elongated side and a second insulative tube extends partially from the hypotenuse to receive the wire-like active electrode therethrough.

In aspects according to the present disclosure, the donut-style tip is made from ceramic.

Provided in accordance with another aspect of the present disclosure is an electrode assembly for an electrosurgical instrument including a housing having an active electrical connector and a return electrical connector configured to operably engage a distal end of an electrosurgical instrument shaft. The housing encapsulates at least a portion of a pair of substantially triangular ground plates including an elongated side and a hypotenuse. The substantially triangular ground plates each including a tip at a distal end thereof configured to mutually support a donut-style insulator. The donut-style insulator is configured to support a wire-like active electrode therearound, a first end of the wire-like active electrode operably connected to the active electrical connector and a second end of the wire-like active electrode operably connected to a tensioning mechanism. The tensioning mechanism cooperates with a tensioning tool to tension the wire-like active electrode about the donut-like insulator during assembly.

In aspects according to the present disclosure, a pair of first and second insulative tubes is configured to house the first and second ends of the wire-like active electrode. In other aspects according to the present disclosure, the first insulative tube extends along an entire length of the elongated side of the pair of the substantially triangular ground plates and the second insulative tube extends partially from the hypotenuse of the pair of substantially triangular ground plates to receive the wire-like active electrode therethrough.

In aspects according to the present disclosure, the tensioning mechanism includes a terminal and an anchoring screw, the anchoring screw configured to secure the terminal against the wire-like active electrode once proper tension has been achieved by rotating the tensioning tool during assembly. In other aspects according to the present disclosure, each tip of each substantially triangular ground plate includes an inwardly extending shaft, the inwardly extending shafts of each tip disposed in registration with one another and configured to mutually support the donut-style insulator.

In aspects according to the present disclosure, the donut-style tip is made from ceramic.

Provided in accordance with another aspect of the present disclosure is a tool for adjusting the tension of a wire-like active electrode of an end effector assembly that includes a handle having a shaft extending therefrom, the shaft adapted to engage a recess defined in a housing of an end effector assembly. A hole is defined in a distal end of the shaft and is configured to receive a wire-like active electrode therethrough. During assembly of the electrode assembly, a distal end of the wire-like active electrode is fed through the hole in the distal end of the shaft, the shaft is then positioned within the recess and the handle is then rotated in a first direction to tension the wire-like active electrode in the end effector assembly.

### BRIEF DESCRIPTION OF THE DRAWINGS

The accompanying drawings, which are incorporated in and constitute a part of this specification, illustrate embodiments of the invention, and together with a general description of the invention given above, and the detailed description of the embodiments given below, serve to explain the principles of the invention.

FIG. 1A is a perspective view of an electrosurgical system including an electrosurgical pencil including a housing having a shaft extending therefrom with an end effector attached to a distal end thereof, the end effector configured for bipolar resection in accordance with an embodiment of the present disclosure;

FIG. 1B is a greatly enlarged view of a proximal end of the end effector and the distal end of the shaft of the electrosurgical pencil housing;

FIG. 2 is a front, top perspective view of the electrosurgical pencil of FIG. 1, with a top-half shell of the housing removed;

FIG. 3 is a perspective view of the plug assembly of FIG. 1, with a top-half shell section removed therefrom;

FIG. 4 is a schematic illustration of the voltage divider network of the present disclosure;

FIG. 5 is a partial, cross-sectional view of an end effector assembly of an electrosurgical pencil, in accordance an embodiment of the present disclosure;

FIG. 6A is an enlarged, top, perspective view of the end effector assembly of the present disclosure;

FIG. 6B is an enlarged, top, exploded view of the end effector assembly of FIG. 6A;

FIG. 6C is a greatly enlarged, exploded view of a tensioning mechanism for use with the end effector assembly according to the present disclosure;

FIGS. 7A and 7B are various views of another embodiment of an end effector assembly in accordance with the present disclosure;

FIGS. 8A and 8B are enlarged, perspective views of a distal end of the end effector assembly of FIGS. 7A and 7B; and

FIGS. 9A and 9B are various views of the end effector assembly of FIGS. 7A and 7B showing a tensioning mechanism cooperating with a tensioning tool to tension an active electrode for tissue treatment.

### DETAILED DESCRIPTION

Particular embodiments of the presently disclosed electrosurgical pencil configured for bipolar resection are described in detail with reference to the drawing figures wherein like reference numerals identify similar or identical elements. As used herein, the term “distal” refers to that portion which is further from the user while the term “proximal” refers to that portion which is closer to the user or clinician. The term “leading edge” refers to the most forward edge with respect to the direction of travel while the term “trailing edge” refers to the edge opposite the leading edge with respect to the direction of travel.

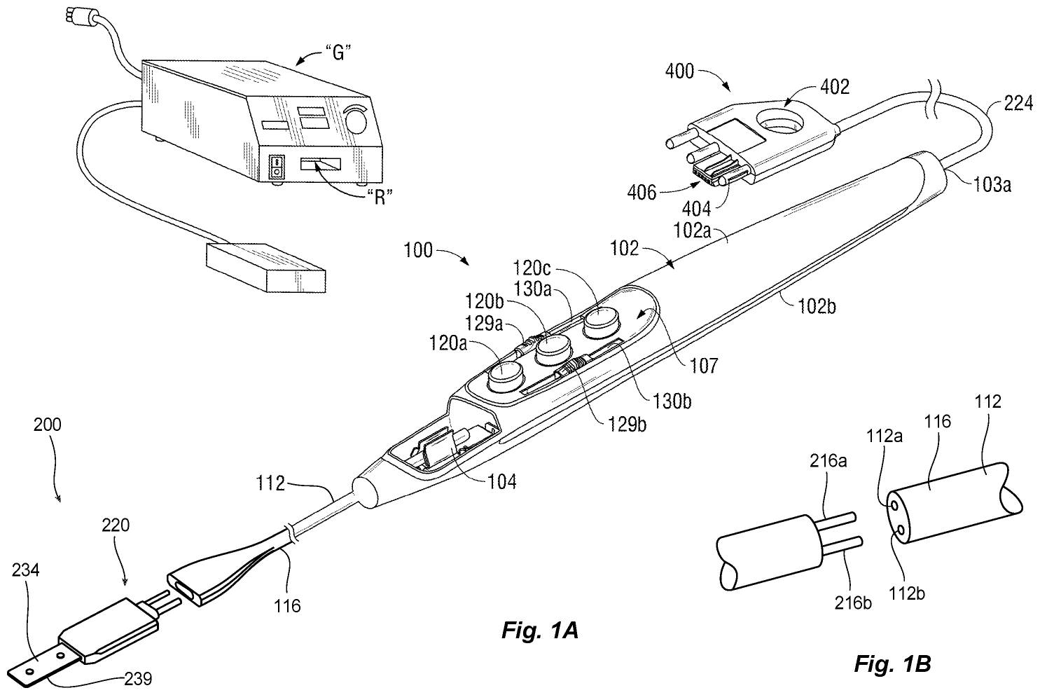

FIGS. 1A-1B sets forth a perspective view of an electrosurgical system including an electrosurgical pencil 100 constructed for bipolar resection in accordance with one embodiment of the present disclosure. While the following description is directed towards electrosurgical pencils for bipolar resection, the features and concepts (or portions thereof) of the present disclosure may be applied to any electrosurgical type instrument, e.g., forc eps, suction coagulators, vessel sealers, wands, etc. The construction, functionality and operation of electrosurgical pencils, with respect to use for bipolar resection, is described herein. Further details of the electrosurgical pencil are provided in commonly-owned U.S. Pat. No. 7,156,842 to Sartor et al.

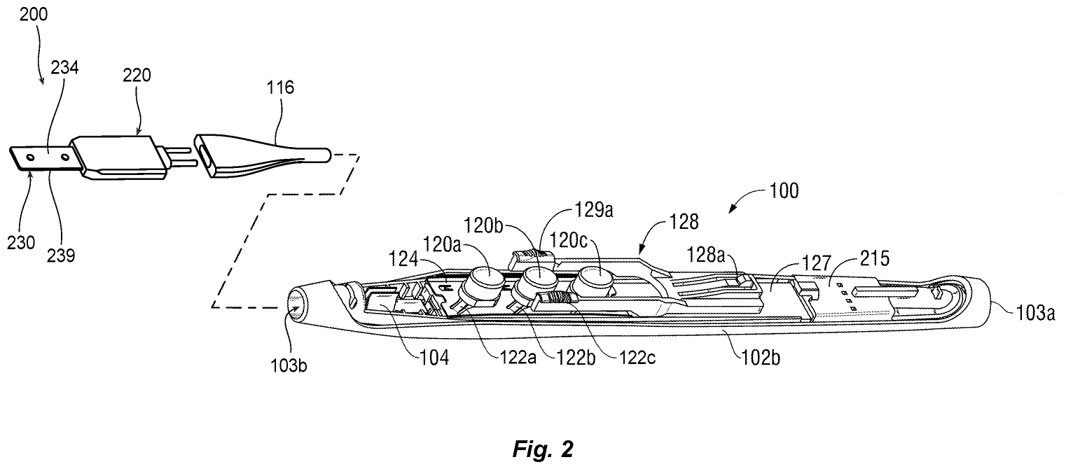

As seen in FIGS. 1A, 1B and 2, electrosurgical pencil 100 includes an elongated housing 102 having a top-half shell portion 102a and a bottom-half shell portion 102b. The elongated housing 102 includes a distal opening 103b, through which a shaft 112 extends, and a proximal opening 103a, through which connecting wire 224 (see FIG. 1A) extends. Top-half shell portion 102a and bottom-half shell portion 102b may be bonded together using any suitable method, e.g., sonic energy, adhesives, snap-fit assemblies, etc.

Electrosurgical pencil 100 further includes a shaft receptacle 104 disposed at a distal end 103b of housing 102 that is configured to receive the shaft 112 of a selectively removable end effector assembly 200. Electrode assembly 200 is configured to electrically connect to generator “G” through various electrical conductors (not shown) formed in the shaft 112, elongated housing 102, connecting wire 224 and plug assembly 400. Generator “G” may be incorporated into the elongated housing 102 and powered by an internal energy supply, e.g., battery or other energy storage device, fuel cell or other energy generation device or any other suitable portable power source.

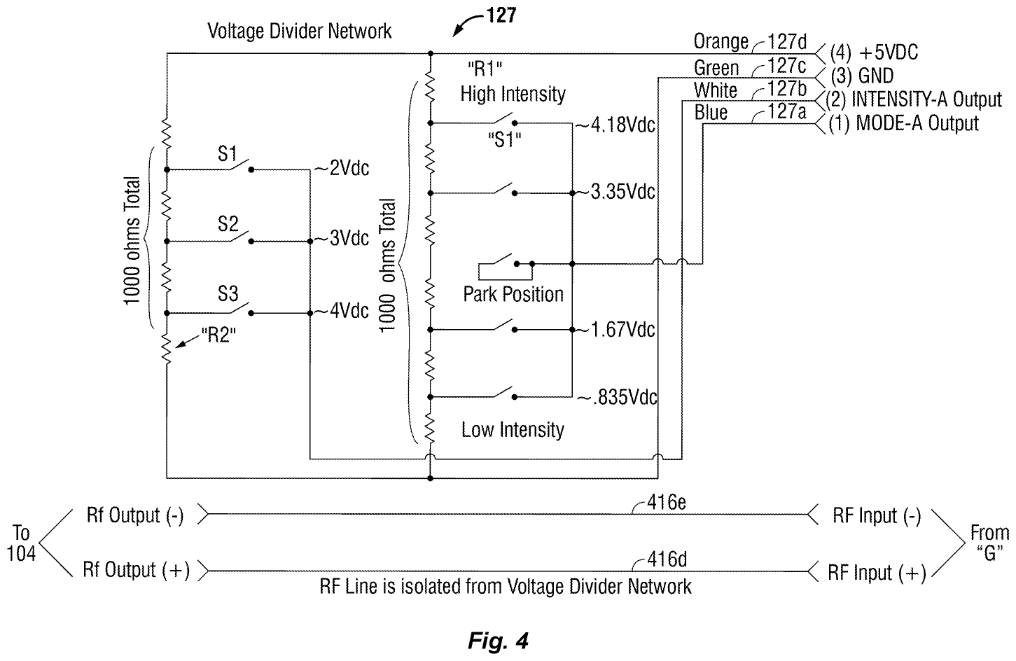

Shaft 112 is selectively retained by shaft receptacle 104 disposed in housing 102. Shaft 112 may include a plurality of conductive traces or wires along the length of the shaft 112. The conductive traces or wires may be fabricated from a conductive type material, such as, for example, stainless steel, or shaft may be coated with an electrically conductive material. Shaft receptacle 104 is fabricated from electrically conductive materials or includes electrically conductive contacts configured to couple with the plurality of conductive traces or wires of the shaft 112. Shaft receptacle 104 is electrically connected to voltage divider network 127 (FIGS. 2 and 4) as explained in more detail below. Conductive traces or wires of the shaft electrically connect to the electrode assembly as explained in more detail below.

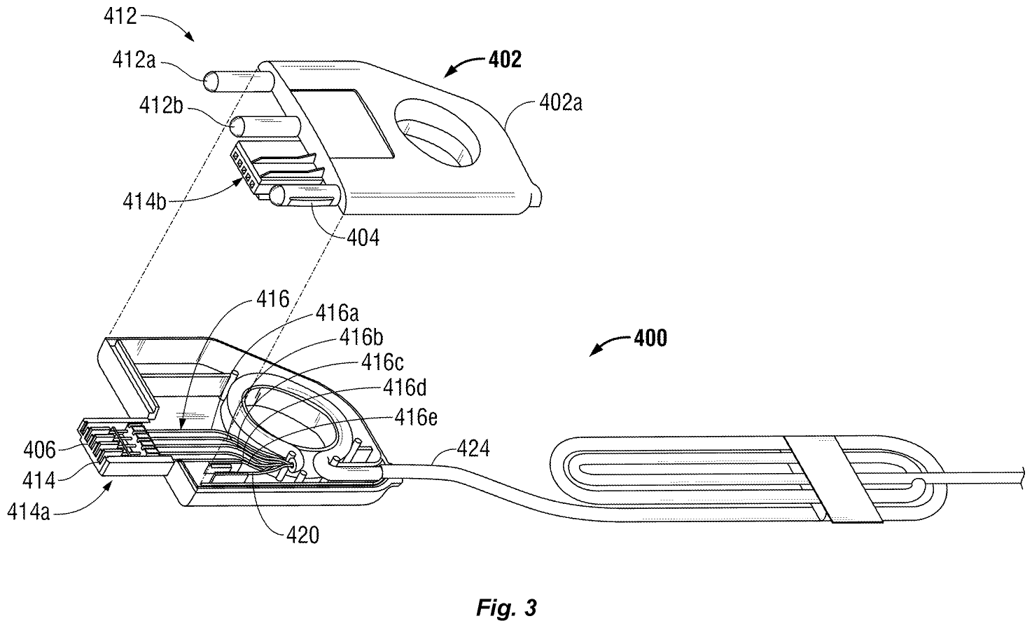

As seen in FIG. 1A, electrosurgical pencil 100 may be coupled to a conventional electrosurgical generator “G” via a plug assembly 400 (see FIG. 3), as will be described in greater detail below.

For the purposes herein, the terms “switch” or “switches” includes electrical actuators, mechanical actuators, electro-mechanical actuators (rotatable actuators, pivotable actuators, toggle-like actuators, buttons, etc.) or optical actuators.

Electrosurgical pencil 100 includes at least one activation switch, and may include three activation switches 120a-120c, each of which extends through top-half shell portion 102a of elongated housing 102. Each activation switch 120a-120c is operatively supported on a respective tactile element 122a-122c provided on a switch plate 124, as illustrated in FIG. 2. Each activation switch 120a-120c controls the transmission of RF electrical energy supplied from generator “G” to bipolar electrodes 138 on electrode face 105 of electrode body 112.

More particularly, switch plate 124 is positioned on top of a voltage divider network 127 (hereinafter “VDN 127”) such that tactile elements 122a-122c are operatively associated therewith. VDN 127 (e.g., here shown in FIG. 2 as a film-type potentiometer) forms a switch closure. For the purposes herein, the term “voltage divider network” relates to any known form of resistive, capacitive or inductive switch closure (or the like) which determines the output voltage across a voltage source (e.g., one of two impedances) connected in series. A “voltage divider” as used herein relates to a number of resistors connected in series which are provided with taps at certain points to make available a fixed or variable fraction of the applied voltage. Further details of electrosurgical pencil control are provided in above-mentioned U.S. Pat. No. 7,503,917 to Sartor et al.

In use, depending on which activation switch 120a-120c is depressed a respective tactile element 122a-122c is pressed into contact with VDN 127 and a characteristic signal is transmitted to electrosurgical generator “G” via control wires 416 (see FIG. 3). In one embodiment, three control wires 416a-416c (one for each activation switch 120a-120c, respectively) are provided. Control wires 416a-416c are electrically connected to switches 120a-120c via a control terminal 215 (see FIG. 2) which is operatively connected to VDN 127. By way of example only, electrosurgical generator “G” may be used in conjunction with the device wherein generator “G” includes a circuit for interpreting and responding to the VDN 127 settings.

Activation switches 120a, 120b, 120c are configured and adapted to control the mode and/or “waveform duty cycle” to achieve a desired surgical intent. For example, a first activation switch 120a can be set to deliver a characteristic signal to electrosurgical generator “G” which, in turn, transmits a duty cycle and/or waveform shape that produces a first desirable resection effect. Meanwhile, second activation switch 120b can be set to deliver a characteristic signal to electrosurgical generator “G” which, in turn, transmits a duty cycle and/or waveform shape that produces a second desirable resection effect.

Finally, third activation switch 120c can be set to deliver a characteristic signal to electrosurgical generator “G” which, in turn, transmits a duty cycle and/or waveform shape that produces a third electrosurgical effect/function. Desirable resection effects may include a mode for bipolar coagulation and/or cauterization with an undeployed blade, a mode for bipolar resection with a partially deployed blade, a mode for bipolar resection with a fully deployed blade, a mode for monopolar resection and a mode for resection with blended energy delivery (monopolar and bipolar modes), as will be described in greater detail hereinbelow.

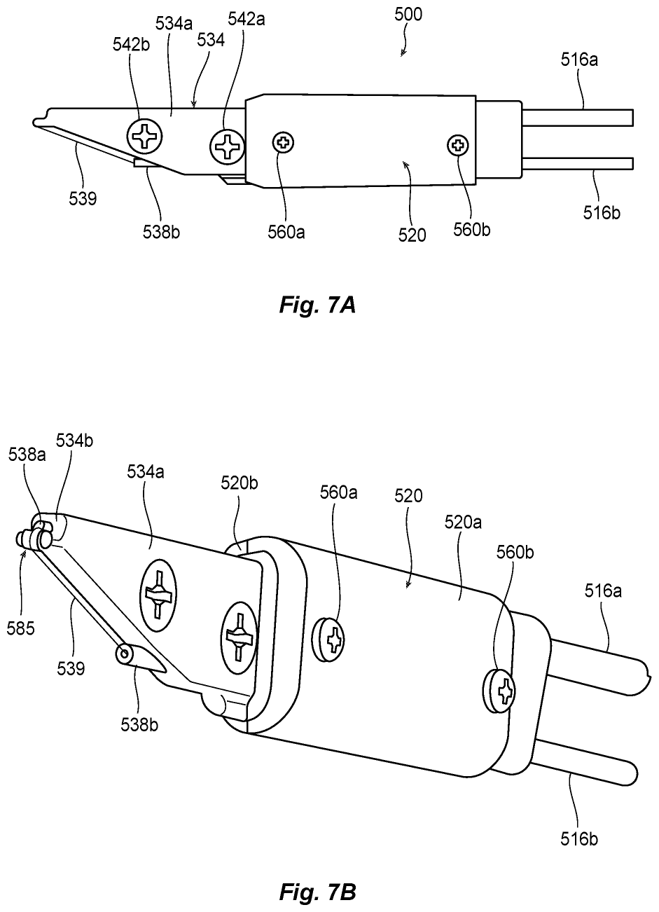

As seen in FIG. 3, fourth and fifth wires (e.g., first RF line 416d and second RF line 416e) are provided and electrically connect to respective active and return electrodes 239, 234 respectively, of the end effector assembly 200 (or end effector assembly 500 as explained in more detail below with respect to FIGS. 7A-9B). Since first RF line 416d and second RF line 416e are directly connected to the end effector assembly 200 first RF line 416d and second RF line 416e bypass the VDN 127 and are isolated from VDN 127 and control wires 416a-416c. By directly connecting the first RF line 416d and second RF line 416e to the end effector assembly 200 (or end effector assembly 500 as explained in more detail below) and isolating the VDN 127 from the RF energy transmission, the electrosurgical current does not flow through VDN 127. This in turn, increases the longevity and life of VDN 127 and/or activation switches 120a, 120b, 120c.

With reference to FIG. 4, VDN 127 is shown and includes a first transmission line 127a configured to operate the various modes of electrosurgical pencil 100; a second transmission line 127b configured to operate the various intensities of electrosurgical pencil 100; a third transmission line 127c configured to function as a ground for VDN 127; and a fourth transmission line 127d which transmits up to about +5 volts to VDN 127.

First RF line 416d and second RF line 416e are isolated from or otherwise completely separate from VDN 127. In particular, first RF line 416d and second RF line 416e extends directly from the RF input or generator “G” to the active electrode and return electrodes of the end effector assembly 200 (or end effector assembly 500 as explained in more detail below).

By way of example only, VDN 127 may include a plurality of resistors “R1” (e.g., six resistors), connected in a first series between third transmission line 127c and fourth transmission line 127d. The first series of resistors “R1” may combine to total about 1000 ohms of resistance. The first series of resistors “R1” are each separated by a first set of switches “S1”. Each switch of the first set of switches “S1” may be electrically connected between adjacent resistors “R1” and first transmission line 127a of VDN 127. In operation, depending on which switch or switches of the first set of switches “S1” is/are closed, a different mode of operation for electrosurgical pencil 100 is activated.

Resection may be performed with electrosurgical energy including waveforms having a duty cycle from about 10% to about 100%. The dual effect of coagulating and cauterizing, as described herein, may be performed with a waveform having a duty cycle from about 10% to about 100%. To increase the depth of coagulation may require a waveform with a duty cycle from about 50% to 100%. It is important to note that these percentages are approximated and may be customized to deliver the desired surgical effect for various tissue types and characteristics.

In one embodiment, the waveforms provided to the bipolar electrosurgical pencil 100 may be dynamically controlled by the generator “G”. For example, the mode of operation provided by switches S1, S2, S3 may indicate a range of operation for the generator “G”. Generator “G” provides a waveform within the specified range of operation wherein the waveform is dynamically changed based on a parameter, wherein the parameter may be related to one of energy delivery, the target tissue and the duration of energy delivery. The parameter may be obtained from a source external to the generator “G”, such as, a measured parameter or clinician provided parameter, or the parameter may include an internal parameter obtained, measured or determined by the generator “G”.

As seen throughout FIG. 2, electrosurgical pencil 100 further includes an intensity controller 128 slidingly supported on or in elongated housing 102. Intensity controller 128 may be configured to function as a slide potentiometer, sliding over and along VDN 127 wherein the distal-most position corresponds to a relative high intensity setting, the proximal-most position corresponds to a low intensity settings with a plurality of intermediate positions therebetween. As can be appreciated, the intensity settings from the proximal end to the distal end may be reversed, e.g., high to low.

The intensity settings are typically preset and selected from a look-up table based on a choice of electrosurgical instruments/attachments, desired surgical effect, surgical specialty and/or surgeon preference, the type of end effector assembly 200 (or end effector assembly 500) and the arrangement of the active and return electrodes 239, 234. The selection of the end effector assembly 200 (or end effector assembly 500) the intensity setting and duty cycle determines the surgical effect. The settings may be selected manually by the user or automatically. For example, the electrosurgical generator “G” may automatically determine the type of end effector assembly 200 (or end effector assembly 500) and a predetermined intensity value may be selected and subsequently adjusted by the user or the electrosurgical generator “G”.

Turning now to FIG. 3, a detailed discussion of plug assembly 400 is provided. Plug assembly 400 includes a housing portion 402 and a connecting wire 424 that electrically interconnects the housing portion 402 and the control terminal 215 in the electrosurgical pencil 100 (see FIG. 2). Housing portion 402 includes a first half-section 402a and a second half-section 402b operatively engageable with one another, e.g., via a snap-fit engagement. First half-section 402a and second half-section 402b are configured and adapted to retain a common power pin 404 and a plurality of electrical contacts 406 therebetween.

Common power pin 404 of plug assembly 400 extends distally from housing portion 402 at a location between first half-section 402a and second half-section 402b. Common power pin 404 may be positioned to be off center, i.e., closer to one side edge of housing portion 402 than the other. Plug assembly 400 further includes at least one a pair of position pins 412 also extending from housing portion 402. Position pins 412 may be positioned between the first half-section 402a and the second half-section 402b of housing portion 402 and are oriented in the same direction as common power pin 404.

A first position pin 412a is positioned in close proximity to a center of housing portion 402 and a second position pin 412b is positioned to be off center and in close proximity to an opposite side edge of housing portion 402 as compared to common power pin 404. First position pin 412a, second position pin 412b and common power pin 404 may be located on housing portion 402 at locations which correspond to pin receiving positions (not shown) of a connector receptacle “R” of electrosurgical generator “G” (see FIG. 1).

Plug assembly 400 further includes a prong 414 extending from housing portion 402. In particular, prong 414 includes a body portion 414a extending from second half-section 402b of housing portion 402 and a cover portion 414b extending from first half-section 402a of housing portion 402. In this manner, when the first half-section 402a and the second half-section 402b are joined to one another, cover portion 414b of prong 414 encloses the body portion 414a. Prong 414 may be positioned between common power pin 404 and first position pin 412a. Prong 414 is configured and adapted to retain electrical contacts 406 therein such that a portion of each electrical contact 406 is exposed along a front or distal edge thereof. While five electrical contacts 406 are shown, any number of electrical contacts 406 can be provided, including and not limited to two, six and eight. Prong 414 may be located on housing portion 402 at a location that corresponds to a prong receiving position (not shown) of connector receptacle “R” of electrosurgical generator “G” (see FIG. 1A).

Since prong 414 extends from second half-section 402b of housing portion 402, housing portion 402 of plug assembly 400 will not enter connector receptacle “R” of electrosurgical generator “G” unless housing portion 402 is in a proper orientation. In other words, prong 414 functions as a polarization member. This ensures that common power pin 404 is properly received in connector receptacle “R” of electrosurgical generator “G”.

Connecting wire 424 includes a power supplying wire 420 electrically connected to common power pin 404, control wires 416a-416c electrically connected to a respective electrical contact 406, and first RF line 416d and second RF line 416e electrically connected to a respective electrical contact 406.

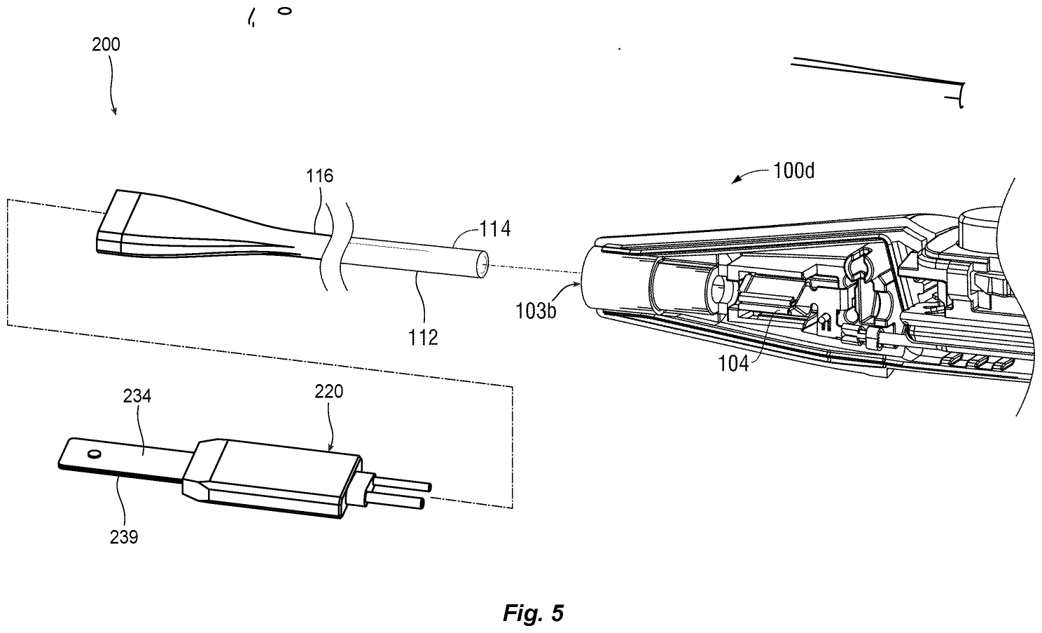

Turning now to FIG. 5, the end effector assembly 200 of electrosurgical pencil 100 is shown wherein a proximal portion 114 of shaft 112 is configured to mechanically and electrically engage shaft receptacle 104. Shaft 112 and shaft receptacle 104 are configured to provide a plurality of suitable electrical connections therebetween to facility the delivery of electrosurgical energy from the electrosurgical generator “G” (See FIG. 1) to the active 239 and return electrode 234 of the end effector assembly 200.

At least a portion of the shaft 112 is inserted into distal opening 103b of the elongated housing 102 to engage shaft receptacle 104. Shaft receptacle 104 is configured to mechanically and electrically couple the shaft 112 to the elongated housing 102. Electrical connections may include one or more electrical connectors (or electrical connector pairs) that connect to the active and return electrodes 239 and 234. Shaft 112 and shaft receptacle 104 may include a locking device, such as, for example, a shaft locking pin that slides into and engages a shaft locking pin receptacle (not explicitly shown). Any suitable securing and/or locking apparatus may be used to releasably secure the shaft 112 to the elongated housing 102. As described herein, the shaft 112 is interchangeable with the elongated housing 102. In other embodiments, shaft 112 is integrated into the elongated housing 102 and is not replaceable.

Turning back to FIG. 1B, a proximal end of the end effector assembly 200 includes a pair of electrical connectors 216a, 216b that is configured to electromechanically couple to a distal end 116 of shaft 112. More particularly, electrical connectors 216a, 216b are configured to mechanically engage respective slots 112a, 112b defined within a distal end of shaft 112. In this manner, the end effector assembly 200 may be interchangeable with shaft 112 and shaft receptacle 104 without having to redesign the interchangeable mechanical connection of the shaft 112 with the shaft receptacle 104 of the electrosurgical pencil 100. Alternatively, shaft receptacle 104 may be designed to selectively accommodate connectors 216a, 216b to provide the proper electrical polarity to end effector assembly 200 upon engagement thereof.

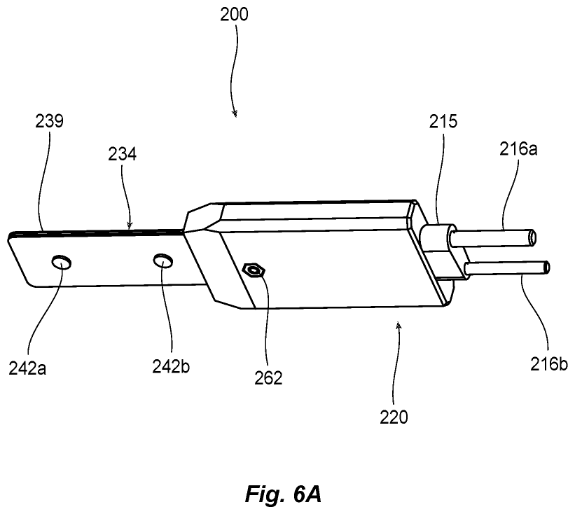

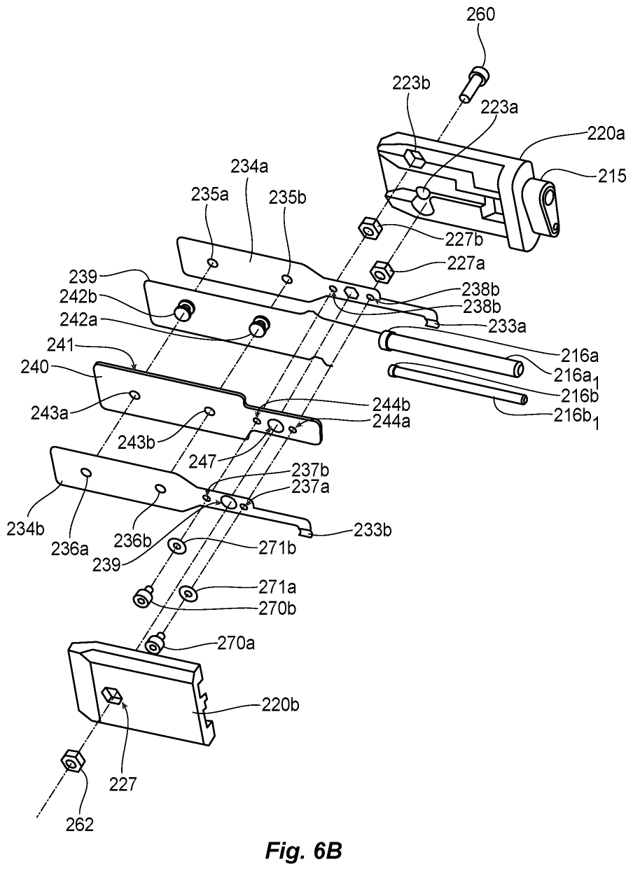

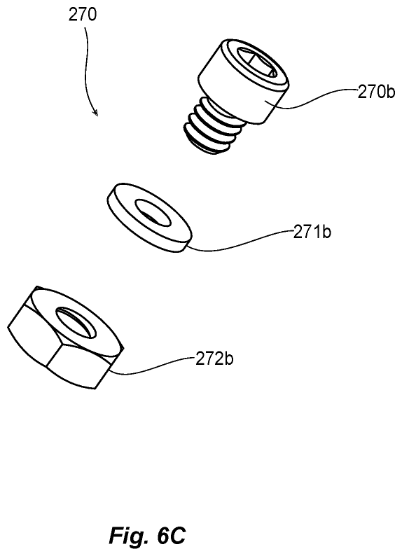

FIGS. 6A-6B show various views of one embodiment of the end effector assembly 200 for use with the electrosurgical pencil 100. End effector assembly 200 includes a housing 220 that is configured to mechanically and electrically couple to a distal end 116 of shaft 112. Housing 220 includes two housing halves 220a, 220b that cooperate to encase electrodes 234a, 234b and an active electrode or cutting wire 239. The housing halves 220a, 220b may be ultrasonically welded together or mechanically engaged in some other fashion, e.g., snap-fit, adhesive, etc. As mentioned above, the distal end 116 of shaft 112 includes a pair of slots 112a, 112b that is configured to mechanical engage proximal connectors 216a, 216b, which, in turn, mechanically and electrically couple to electrodes 234a, 234b and wire 239.

The pair of housing halves 220a, 220b encapsulate the return electrodes 234a, 234b, an insulative core 240 and the respective distal ends 216a1, 216b1 of the connectors 216a, 216b. Housing halves 220a, 220b are secured via screw 260 and nut 262. Nut 262 may be recessed within a nut cavity 227 defined within an outer facing side of housing half 220b. Screw 260 may be recessed within housing half 220a. More particularly, each return electrode 234a, 234b affixes to a respective opposing side of the insulative core 240 and is held in place via a pair of rivets 242a, 242b. Each rivet 242a, 242b engages a corresponding aperture defined in the insulative core 240 (namely, apertures 243a, 243b) and each electrode 234a (namely, apertures 235a, 235b), 234b (namely, apertures 236a, 236b). The insulative core 240 may be made from any insulative material, e.g., ceramic, and is dimensioned slightly larger than the dimensions of respective return electrodes 234a, 234b.

Respective proximal ends 233a, 233b of each return electrode 234a, 234b is configured to electrically engage connector 216b. Proximal ends 233a, 233b may include geometry to facilitate connection to the connector 216b, e.g., an arcuate flange or other mechanical interface.

Wire 239 is configured to partially seat within a slot 241 defined along the outer peripheral edge of insulative core 240. Part of the wire 239 remains exposed to allow electrically cutting (as explained in more detail below). Wire 239 is configured to electrically connect to connector 216a (e.g., active electrode) which supplies a cutting current when the electrosurgical pencil 100 is activated. Wire 239 may be made from tungsten or any other type of material commonly used in the art.

During assembly and once wire 239 is seated within slot 241, the wire 239 is tensioned utilizing a tensioning mechanism 270. Tensioning mechanism 270 includes a pair of bolts 270a, 270b, a corresponding pair of washers 271a, 271b and a corresponding pair of nuts 272a, 272b (See FIG. 6C). Wire 239 is fed from connector 216a, between bolt 270a and nut 272a pair, and atop washer 271a and then around the distal-most edge of the insulative core 240 to be secured between bolt 270b and nut 272b pair atop washer 271b. Each washer 271a, 271b crimps the wire 239 to the face of the respective nut 272a, 272b. Various types of washers 271a, 271b may be used to facilitate this purpose, e.g., spring washers or wave washers. Pinching the wire 239 against the nuts 272a, 272b via the washers 271a, 271b provides tension to the wire 239 and secures the wire 239 within the slot 241. During assembly and testing, the bolts 270a, 270b may be tightened as necessary to provide a requisite amount of tension to wire 239. The addition of a washer 271a, 271b provides consistent and robust tensioning that may be modified as necessary for testing and final assembly.

Each bolt 270a, 270b engages a corresponding aperture defined in the core 240 (namely, apertures 244a, 244b) and each electrode 234a (namely, apertures 237a, 237b), 234b (namely, apertures 238a, 238b). Nuts 272a, 272b may be seated within respective nut cavities 223a, 223b defined within housing half 220a.

Once assembled, end effector assembly 200 may be selectively attached to the distal end 116 of the shaft 112 as explained above. A proximal end of the housing 220 (once assembled) may include a proximal housing support 215 that engages and supports the connectors 216a and 216b. Proximal housing support 215 may be tapered to facilitate assembly and orientation of the end effector assembly 200 with the shaft 112 or pencil housing 102.

As mentioned above, the wire 239 may be made from any suitable conductive material such as tungsten, surgical stainless steel, etc. Tungsten is particularly favored since various geometries for the wire 239 may be easily 3D printed providing additional robustness over traditional wire designs while offering an optimized surface area to increase cutting efficiency. Moreover a sheet including a plurality of tungsten wires 239 may be 3D printed to facilitate the manufacturing process. Moreover, multiple geometries may be easily integrated with the mating geometry of the various mechanical interfaces staying the same. The exposed edge (not explicitly shown) of wire 239 is configured for cutting and is designed to concentrate electrosurgical energy to increase cutting efficiency.

The return electrodes 234a, 234b are made from a conductive material and insulated from the wire 239 via the insulative core 240. As mentioned above, the insulative core 240 may be made from a material that provides good thermal and non-conductive properties. Each return electrode 234a, 234b provides a return path for the electrosurgical energy from the wire 239 such that the circuit is completed.

Turning now to FIGS. 7A-9B, another embodiment of an end effector assembly is shown and designated end effector assembly 500. End effector 500 is a single-sided treatment or cutting tool inasmuch as the active electrode 539 is exposed on one side (e.g., bottom or exposed side 539a) of the end effector assembly 500.

End effector assembly 500 is similar to the end effector assembly 200 described above and, as such, only those details necessary for a complete understanding of end effector assembly 500 are discussed herein. End effector 500 includes a housing 520 having housing halves 520a 520b that cooperate to encapsulate the active electrode 539, a portion of a return electrode 534 and a tensioning mechanism 570. One or more bolt and nut arrangements 560a, 560b may be utilized to secure the two housing halves 520a, 520b together. The return electrode 534, in turn, includes a pair of opposing ground plates 534a, 534b that cooperate to encapsulate hypotubes 538a, 538b and active electrode 539 as explained in more detail below.

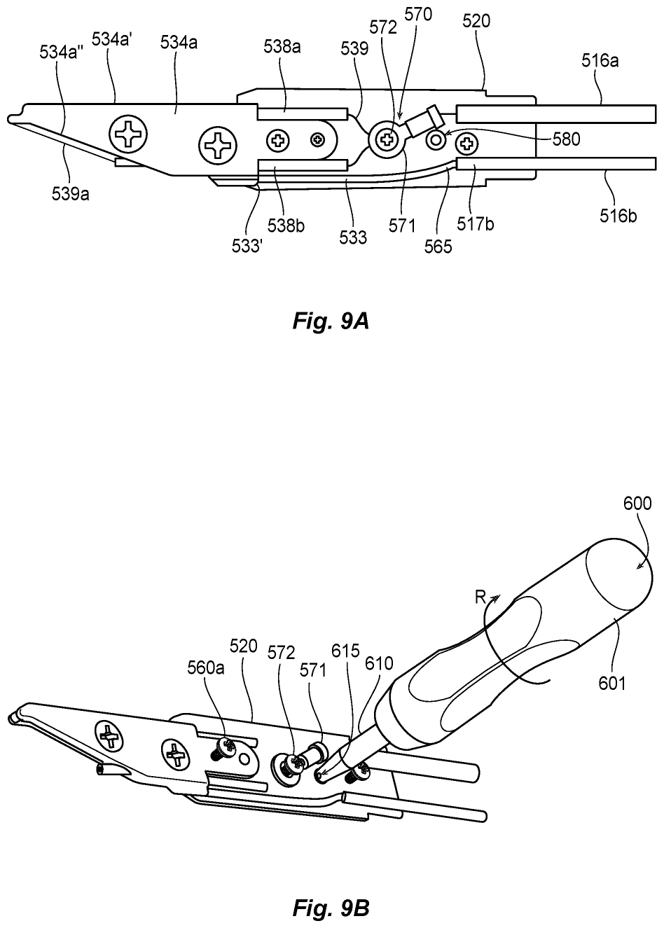

End effector 500 includes a donut-style plunging tip 585 configured to guide active electrode (or active wire) 539 therearound for engagement with the tensioning mechanism 570 as described in more detail below with respect to FIGS. 9A and 9B. Alternatively, a tensioning mechanism similar to tensioning mechanism 270 described above may be employed to tension active electrode 539. Donut-style plunging tip 585 may be made from ceramic or any other type of durable material that both electrically isolates the active electrode 539 from the return electrode 534 and provides the necessary rigidity to support the active electrode 539 for tissue treatment (e.g., cutting).

Donut-style plunging tip 585 guides the active electrode 539 therearound to transition the active electrode 539 distally to proximally to permit the active electrode 539 to treat tissue (e.g., cut tissue) on the exposed side 539a of the active electrode 539. More particularly, and as best shown in FIG. 9A, active electrode 539 engages active electrical connector 516a at a distal end thereof. Active electrode 539 may be attached to the active electrically connector 516a in any way known in the art. Active electrode 539 transitions through the tensioning mechanism 570 (explained in detail below) and is then fed through hypotube 538a, to and around donut-style plunging tip 585, to second hypotube 538b and back into engagement with the tensioning mechanism 570. As explained in detail below, tensioning mechanism 570 provides the necessary tension onto active electrode 539 to insure effective tissue treatment (e.g., cutting). The donut-style plunging tip 585 provides the necessary rigidity and electrical isolation of the active electrode 539 at the distal end of the end effector assembly 500 to insure tissue treatment (e.g., cutting).

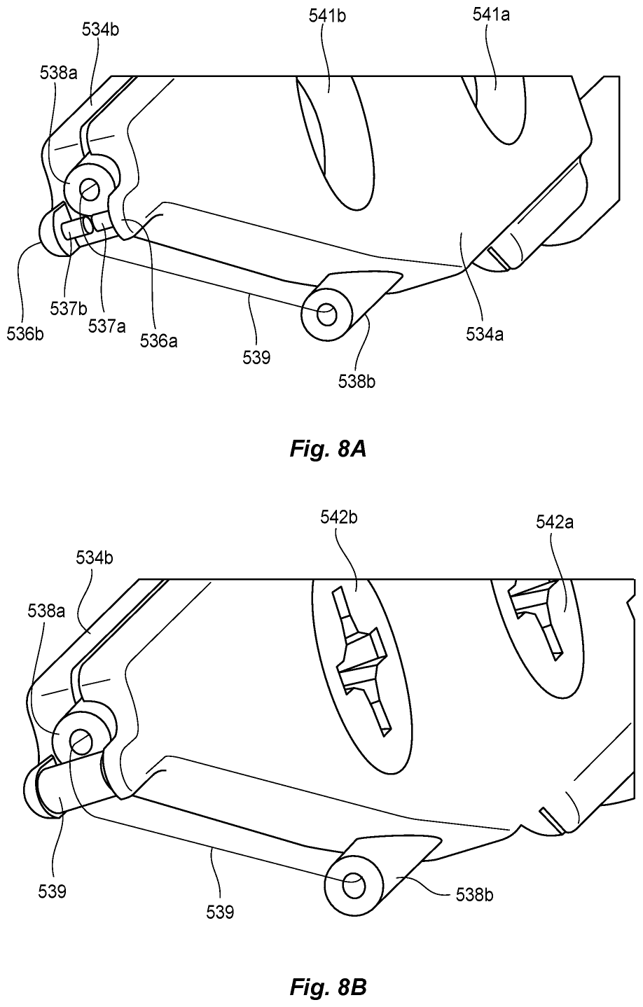

Return electrode 534 includes the pair of ground plates 534a, 534b that cooperate to encapsulate the active electrode 539 and the hypotubes 538a, 538b therein. Each ground plate 534a, 534b includes a respective tip 536a, 536b that projects from a distal end thereof. Each tip 536a, 536b supports a respective shaft 537a, 537b that mutually project inwardly in substantial registration with one another and are configured to support the donut-style plunging tip 585 thereon (See FIGS. 8A and 8B). Donut-style plunging tip 585 includes a central aperture (not shown) that mounts atop each respective shaft 537a, 537b similar to a toilet paper holder at assembly.

Ground plate 534a includes a pair of coined recesses 541a, 541b defined therein configured to receive a respective set screw 542a, 542b. Each set screw 542a, 542b engages a corresponding thread (not shown) defined in ground plate 534b. The set screw 542a, 542b and thread arrangements cooperate to secure the ground plates 534a, 534b together during assembly. Likewise nut and bolts arrangements 560a, 560b (or the like) engage the two housing halves 520a, 520b to one another about the ground plates 534a, 534b, tensioning mechanism 570 and active electrode 539.

Return electrode 534 (e.g., ground plates 534a, 534b) connects to a distal end 533′ of a return lead 533 that ultimately connects to the return electrical connection 516b by a threaded or other suitable connection disposed at a proximal end thereof. For example, distal end 517b of the return electrical connector 516b may be threaded to engage a corresponding threaded connection 565 disposed at the proximal end of the return lead 533 (FIG. 9A).

Return electrode 534 (e.g., ground plates 534a, 534b) provide a large, robust surface area of electrical return for the active electrode 539 when engaging tissue to facilitate bipolar treatment thereof. In addition, the size of the return electrode 534 (e.g., ground plates 534a, 534b) provides better heat capability to the end effector assembly 500 which reduces the chances of eschar buildup. The return electrode 534 (e.g., ground plates 534a, 534b) may be substantially triangular in shape to facilitate orientation of the exposed portion 539a of the active electrode 539 during use (FIG. 9A). The substantial triangular configuration of each ground plate includes an elongated side and a hypotenuse, e.g., an elongated side 534a′ and a hypotenuse 534a″ of ground plate 534a.

FIGS. 9A-9B show the tensioning mechanism 570 along with a tensioning tool 600 configured to facilitate tensioning the active electrode 539 about the donut-style plunging tip 585. As discussed above, the active electrode 539 is operably engaged at a first end to the active electrical connector 516a which is then fed through a terminal 571 of the tensioning mechanism 570, through hypotube 538a, around the donut-style plunging tip 585, through hypotube 538b, and back to operably anchor to the terminal 571 of the tensioning mechanism 570 via an anchoring screw 572.

During assembly, the active electrode 539 is fed through end effector assembly 500 in the manner described above with the tensioning mechanism 570 disposed in a first, pre-anchored position (anchoring screw 572 is spaced relative to terminal 571) to facilitate engagement with the active electrode 539. Once the active electrode is oriented about the end effector assembly 500 as described above, the tensioning tool 600 is introduced to engage the active electrode for tensioning. More particularly, tool 600 includes a handle 601 and shaft 610 that extends therefrom. The shaft 610 includes a hole 615 defined in a distal end thereof that is configured to engage the distal end of the active electrode 539 after it is routed through the end effector 500.

A recess 580 is defined in housing half 520b and is configured to allow the distal end of the tensioning tool to seat therein to facilitate engagement with the distal end of the active electrode 539. While seated in the recess 580, tool 600 can be rotated in the direction “R” to tension the active electrode 539 about the donut-style plunging tip 585. Once properly tensioned, the anchoring screw 572 may be tightened to secure the active electrode 539 in tension and the tool 600 may be removed. As can be appreciated, the exposed portion 539a of the active electrode 539 is now ready for tissue treatment upon activation thereof. Energy from the electrosurgical generator “G” is supplied to the active electrode 539 to treat tissue and returned through ground plates 534a, 534b to complete the bipolar electrical circuit.

The various embodiments disclosed herein may also be configured to work with robotic surgical systems and what is commonly referred to as “Telesurgery.” Such systems employ various robotic elements to assist the clinician and allow remote operation (or partial remote operation) of surgical instrumentation. Various robotic arms, gears, cams, pulleys, electric and mechanical motors, etc. may be employed for this purpose and may be designed with a robotic surgical system to assist the clinician during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of clinicians may prep the patient for surgery and configure the robotic surgical system with one or more of the instruments disclosed herein while another clinician (or group of clinicians) remotely controls the instruments via the robotic surgical system. As can be appreciated, a highly skilled clinician may perform multiple operations in multiple locations without leaving his/her remote console which can be both economically advantageous and a benefit to the patient or a series of patients.

For a detailed description of exemplary medical work stations and/or components thereof, reference may be made to U.S. Patent Application Publication No. 2012/0116416, and PCT Application Publication No. WO2016/025132, the entire contents of each of which are incorporated by reference herein.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. An electrode assembly for an electrosurgical instrument, comprising:
a housing including an active electrical connector and a return electrical connector configured to operably engage a distal end of an electrosurgical instrument shaft, the housing encapsulating a pair of elongated ground plates and a pair of insulative tubes configured to house first and second ends of a wire-like active electrode, the wire-like active electrode operably coupled at the first end to the active electrical connector, the elongated ground plates each including a tip at a distal end thereof configured to mutually support a donut-style insulator, the donut-style insulator configured to support the wire-like active electrode therearound; and
a tensioning mechanism configured to operably engage the second end of the wire-like active electrode, the tensioning mechanism cooperating with a tensioning tool to tension the wire-like active electrode about the donut-like insulator during assembly.

2. The electrode assembly of claim 1 wherein the tensioning mechanism includes a terminal and an anchoring screw, the anchoring screw configured to secure the terminal against the wire-like active electrode once proper tension has been achieved by rotating the tensioning tool during assembly.

3. The electrode assembly of claim 1 wherein each tip of each ground plate includes an inwardly extending shaft, the inwardly extending shafts of each tip disposed in registration with one another and configured to mutually support the donut-style insulator.

4. The electrode assembly of claim 1 wherein the pair of ground plates are substantially triangular to expose a portion of the wire-like active electrode for tissue treatment.

5. The electrode assembly of claim 1 wherein a first insulative tube extends along an entire length of one side of the pair of ground plates to limit exposure of the wire-like active electrode.

6. The electrode assembly of claim 1 wherein the pair of ground plates are substantially triangular including an elongated side and a hypotenuse and wherein a first insulative tube extends along an entire length of the elongated side and a second insulative tube extends partially from the hypotenuse to receive the wire-like active electrode therethrough.

7. The electrode assembly of claim 1 wherein the donut-style tip is made from ceramic.

8. An electrode assembly for an electrosurgical instrument, comprising:
a housing including an active electrical connector and a return electrical connector configured to operably engage a distal end of an electrosurgical instrument shaft, the housing encapsulating at least a portion of a pair of substantially triangular ground plates including an elongated side and a hypotenuse, the substantially triangular ground plates each including a tip at a distal end thereof configured to mutually support a donut-style insulator, the donut-style insulator configured to support a wire-like active electrode therearound, a first end of the wire-like active electrode operably connected to the active electrical connector and a second end of the wire-like active electrode operably connected to a tensioning mechanism, the tensioning mechanism cooperating with a tensioning tool to tension the wire-like active electrode about the donut-like insulator during assembly.

9. The electrode assembly of claim 8 wherein a pair of first and second insulative tubes is configured to house the first and second ends of the wire-like active electrode.

10. The electrode assembly of claim 9 wherein the first insulative tube extends along an entire length of the elongated side of the pair of the substantially triangular ground plates and the second insulative tube extends partially from the hypotenuse of the pair of substantially triangular ground plates to receive the wire-like active electrode therethrough.

11. The electrode assembly of claim 8 wherein the tensioning mechanism includes a terminal and an anchoring screw, the anchoring screw configured to secure the terminal against the wire-like active electrode once proper tension has been achieved by rotating the tensioning tool during assembly.

12. The electrode assembly of claim 8 wherein each tip of each substantially triangular ground plate includes an inwardly extending shaft, the inwardly extending shafts of each tip disposed in registration with one another and configured to mutually support the donut-style insulator.

13. The electrode assembly of claim 8 wherein the donut-style tip is made from ceramic.

14. A tool for adjusting the tension of a wire-like active electrode of an end effector assembly, comprising:
a handle having a shaft extending therefrom, the shaft adapted to engage a recess defined in a housing of an end effector assembly; and
a hole defined in a distal end of the shaft, the hole configured to receive a wire-like active electrode therethrough, wherein during assembly of the electrode assembly, a distal end of the wire-like active electrode is fed through the hole in the distal end of the shaft, the shaft is then positioned within the recess and the handle is then rotated in a first direction to tension the wire-like active electrode in the end effector assembly.
